(19) United States
(12) Patent Application Publication
(10) Pub. No.: US 2005/0019394 A1
(43) Pub. Date: Jan. 27, 2005

(54) Carrageenan-containing composition with improved gelatinisation properties

(76) Inventors: Huilin Xia, Stuttgart (DE); Hans-Gerhard Fritz, Uhingen (DE); Karin Born, Wiesbaden (DE); Fabianne Bart, Carentan (FR)

Correspondence Address: Gary M. Nath;NATH & ASSOCIATES PLLC, 6th Floor, 1030 15th Street, N.W., Washington, DC, 20005, US

(21) Appl. No.: 10/817,947

(22) Filed: Apr. 6, 2004

Related U.S. Application Data
(63) Continuation of application No. PCT/EP02/11863, filed on Oct. 23, 2002.

(30) Foreign Application Priority Data
Oct. 23, 2001 (DE) 101 52 140.5

(51) Int. Cl. A61K 38/00; A61K 31/737; A61K 9/20

(52) U.S. Cl. 424/464; 514/54; 514/2; 514/60

(57) ABSTRACT

The invention relates to carrageenan-containing compositions with improved gelling properties, syneresis ≦3.0 wt. % and breaking strength improved by at least 20% compared to the carrageenan-containing starting material. Said composition contains at least 60 wt. % of carrageenan, preferably k-carrageenan, and a hydrocolloid or protein starch or mixtures thereof. The invention also relates to a method for improving the gelling properties of a carrageenan-containing composition, comprising humidifying the starting material, preferably with water, mixing it at elevated temperatures and conditions of high pressures and high shearing strains, and finely cooling the output material before comminuting it. The invention also relates to the use of the composition in nutriment and pharmaceutical formulations, preferably as texturing agent, viscosifier, gelling agent, film forming agent, Theological aid or stabilizer. The compositions have improved gelling consistency, reduced syneresis and accelerated gelation capacity and, in powder form, have excellent dispersing capacity in solutions.

The present invention relates to a carrageenan-containing composition having improved gelatinisation properties, a process for improving the gelatinisation properties of a carrageenan-containing composition and the use of said composition.

Carrageenans are polysaccharides of galactose moieties having different degrees of sulfation between 15 and 40%. As typical marine hydrocolloids, they are usually obtained from red algae and used mainly in the food industry as gelatinisation agents or thickeners with thermoreversible characteristics.

Even more than 600 years ago, the so-called “Irish moss” in carrageenan was used for medical purposes and as a food component in Carraghen on the coast of Southern Ireland. “Irish moss” was known for its unique property of thickening milk. Accordingly, it was also used in coastal regions of Normandy and Brittany. For example, cakes were prepared with bleached lichen or so-called “goémon blanc” by simply boiling algae in milk.

From the middle of the 20^{th} century onwards, such extracts have been prepared on an industrial scale. While “Irish moss” was used originally, carrageenans are obtained from numerous red algae today.

In general, two kinds of carrageenans can be distinguished: traditionally purified extracts and semi-purified carrageenans (processed Eucheuma Seaweed, P.E.S.) which have found their way into the food industry only recently. According to the limited properties of P.E.S., the following statements exclusively relate to carrageenan extracts purified in the traditional manner.

The extensive carrageenan family is highly diverse and may generally be divided into three “ideal” main groups, namely the iota, kappa and lambda carrageenans, gel-forming carrageenans being represented exclusively by iota and kappa carrageenans.

These carrageenan species are linear polymers consisting of two repeating carrabiose units:

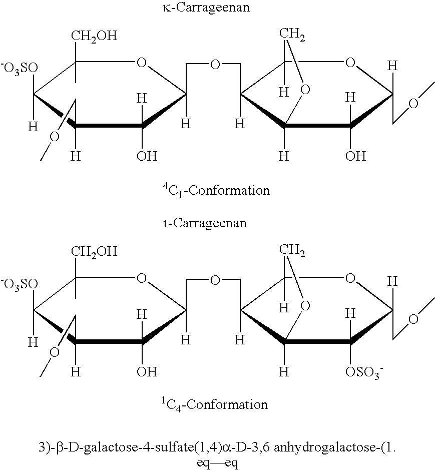

Since the anhydrogalactose is present in a ¹C₄-conformation, all glycosidic bonds have a diequatorial orientation. The only difference between iota and kappa carrageenans is esterification with sulfuric acid: in such cases, the kappa carrageenan is anchored exclusively to the hydroxyl group in position C-4 of the galactosyl radical and has a sulfate content of approx. 25 to 30%. In iota carrageenan, in addition to the C-4 position, the hydroxyl group is also esterified in the C-2 position of the anhydrogalactosyl radical, which is why iota carrageenans have a sulfate content of 28 to 35%.

The glucosidic bonds with diequatorial orientation permit formation of molecule chains in a double helix shape, this orderly structure being able to convert to binding zones in the presence of special cations, thus permitting gelatinisation.

The iota carrageenan gel is transparent, flexible and relatively weak, and the loose network may be destroyed easily by mechanical forces. However, it reverts to its original structure comparatively fast as soon as these mechanical influences discontinue (thixotropy).

In kappa carrageenans, gelatinisation is increased especially by potassium ions, where even comparatively low concentrations induce gelatinisation. Owing to their small ion size, potassium ions may be intercalated into the coils of polysaccharides and partially neutralise the sulfate groups there, at which, the double helices combine into aggregates and as a result can form a strong gel.

Industrial harvesting of carrageenans generally makes use of alkaline extraction followed by the combined separation steps of centrifugation and/or filtration. The comparatively clear, carrageenan-containing solution thus obtained is then typically precipitated with an alcohol, washed, pressed out, dried and then ground.

The flexible products thus obtained have specific rheological characteristics and may be used either alone or in combination with other hydrocolloids such as locust bean flour in many different areas of application.

In the food sector, for example:
for stabilising chocolate drinks and creams
in desserts such as thickened milk products, cakes, layered desserts and mousse

In meat and fish products:
for injection into ham and poultry meat
for fat reduction or as a fat substitute, for example in hamburgers;
in tinned food, for example in combination with locust bean flour, also for animal fodder

In instant products:
for cake bottom layers
in desserts, cream cakes and pastry creams
in desserts on water basis (for example jello) and coatings/frostings

In ice-cream:
usually in combination with guar flour, alginates and locust bean flour

In non-food applications:
in toothpaste and cosmetics
in agents to improve the air
in pharmaceutical products.

On the whole, however, carrageenans are traditionally used in gels containing water. For example, carrageenans are used in aspic, but also as gels in the tinned food industry, for preparing animal fodder and in connection with boiled and sliced meat products (for example cold cuts) in order to protect them against loss of moisture, to improve the result of boiling, to make them easy to cut and, not least, to make them more palatable.

Especially for the meat and sausage products last mentioned, the water retention ability is significant, the continuous improvement of the cutting characteristics being of particular importance.

From the prior art, numerous publications and patents are known which deal with the extrusion of hydrocolloids, carrageenans, however, are mentioned in only a few.

In this connection, WO 99/47 249 titled “Extruded hydrocolloid granules with improved wettability and a process for producing same”, for example, describes improved dispersing behaviour for a guar product and enhanced humidifying properties in aqueous media if it is ground with a solid, non-ionic humidifying agent in the dry state and then extruded.

Wedlock et al. (“Pregelatinised galactomannans—Properties and applications; 1983) found that blends of guar and locust bean flour have increased basic viscosities in cold water and, jointly with xanthan, are capable of developing a gel.

U.S. Pat. No. 4,859,484 protects “Processed starch-gum blends” which show viscosity characteristics for a mixture of a hydrocolloid and starch in water which correspond to those of the raw material if used in a 10 to 20% excess. Such improved properties are attributed to intimate mixing as takes place in an extrusion process.

On the whole, macromolecules are practically incapable of withstanding high shear forces, high temperatures and conditions of elevated pressure as occur in the course of extrusion processes. The decrease of the molecular weight resulting from such influences always has a negative impact on the properties of the molecule, for example the strength of the gel.

Single- and double-screw extruders contain an extruder cylinder as a component in which one or two extruder screws rotate. The extruder is equipped with heating systems divided into sectors by means of which an axial temperature profile may be applied to the extruder cylinder. The heating temperatures of the cylinder sectors are controlled individually and may be adjusted separately in accordance with the requirements of the process technology. The cylinder/screw system receives the starting mixture, conveys it into the direction of the screw tip, heats it in the course of heat transfer and dissipation processes and builds up mass pressure in the medium. By the combined action of the screw(s) and the cylinder, the starting materials are dispersed, mixed and their structure is modified by the action of shear forces, pressure and temperature. Such structural changes are shown, for example, by a change from the solid state to a melt, in viscous flow properties of the resulting plasticised material and a change in consistence (texturing). After leaving the extruder, the extrudate is converted back to the solid state by forced cooling.

In particular, the technology of extrusion is used to prepare so-called texturised food products such as breakfast cereals and snack products, but also for the preparation of instant products. An analogous technology is also used to prepare starch products which are soluble in cold water as well as instant products containing starch.

In addition, the extrusion technology is the present state of the art as far as the intimate mixing of components to be extruded is concerned, with synergy effects being in particular achieved, for example, with regard to an increased viscosity of the components subjected to intimate mixing and modification by shear forces which is noticeably higher than that of the starting materials simply mixed with each other.

In view of the state of the art as described above, it was therefore the object of the present invention to provide a carrageenan-containing composition with improved gelatinisation characteristics.

This object was achieved with a corresponding carrageenan-containing composition having a syneresis of ≦3.0 wt.-% and an improved breaking strength of at least 20% when compared with the carrageenan-containing starting material.

In the course of solving this problem, a process for treating a carrageenan-containing composition to improve its gelatinisation properties is also provided, where the starting material being first humidified, preferably with water, then subjected to intensive shearing and mixing at elevated temperatures and under the conditions of an increased pressure, which is preferably carried out by shearing and mixing aggregates, and finally, the mass being cooled and ground. The composition obtained by this process is another subject matter of the present invention.

A further subject matter of this invention is the use of the novel carrageenan-containing composition with improved gelatinisation properties, said composition being used in formulations in the food and/or the pharmaceutical industry, especially in gels containing water.

Contrary to previous experience from the prior art, it was a great surprise that even extreme conditions such as elevated mass pressures, temperatures and high shear stresses cannot destroy the carrageenan structures and that, moreover, the carrageenan-containing composition of the invention even has an increased gel strength, a reduced syneresis of the gel, an accelerated gelatinisation capacity as well as a significantly improved dispersibility of the composition in powder form in solutions. It is also possible with the aid of the carrageenan-containing compositions of the invention to obtain blends with other polymers whose characteristics are clearly superior to those of comparable powders/powder blends. None of this was to be expected to such a pronounced degree.

As a matter of principle, any suitable carrageenan-containing material may be used as raw material for the present invention. In accordance with the invention, however, it has turned out to be advantageous if the carrageenan-containing composition preferably contains κ-carrageenan.

Likewise, it has been shown to be advantageous when, in connection with the present invention, the syneresis of the carrageenan-containing composition is ≦2.0 wt.-% and/or the composition has an improved breaking strength which is increased by at least 40% when compared with the carrageenan-containing starting material.

A composition with a carrageenan content of at least 60% is also preferred.

A special variation of the present invention provides a composition which, in addition to the carrageenan component, contains a starch and/or a hydrocolloid and/or a protein, with each being able to be subjected to pre-treatment.

The starch used is preferably derived from corn, maize, potatoes, wheat, tapioca, rice or mixtures thereof.

It is also preferred if the hydrocolloid component is one of the series of guar, locust bean flour, karaya, konjac, cellulose, cellulose ether, microcrystalline cellulose, xanthan, pectin, alginates, agar or any mixture thereof.

The third also preferred additional component of the invention, the protein, may quite generally within the present invention be of any vegetable or animal origin, it may be derived from leguminous plants or be a soy protein which is particularly well suited.

On the whole, the content of the additional components in the composition of the invention is not critical, but a total content of not more than 50 wt.-% based on the total weight is recommended, a content between 3 and 40 wt.-% being regarded as particularly suitable.

Finally, it is preferred to use a composition in the invention which is a product subjected to treatment under high shear forces and high pressure, as is the case in an extrusion process with single- or twin-screw extruders, for example.

The carrageenan-containing composition of the invention has improved properties, especially with a view to its application in the food sector, such as improved gel strength, improved interaction with other (bio) polymers, improved gel syneresis and a markedly improved dispersibility in powder form, and—very surprisingly—may be obtained by an extrusion process which is known to take place under high shear forces, high pressure and high temperatures in that part of the process where the mass is conveyed.

For this reason, the present invention also comprises a process for improving the gelatinisation properties of a carrageenan-containing composition wherein the raw material is humidified, preferably with water, in a first process step, this mixture is then subjected to intensive shearing and mixing at elevated temperatures under conditions of increased pressure, with this taking place under high shear stresses and preferably generated in shearing and mixing aggregates such as single- or twin-screw extruders and in which finally the mass thus obtained is cooled and ground. In general, this process influences the characteristics of the composition of the invention by controlling individual process parameters, especially the moisture content of the material to be processed, the temperature profile of the extruder as well as the feed rate of the starting material and the rotational speed of the kneader (extruder).

Preferably, the starting material contains at least 60 wt.-% of carrageenans and especially at least 60 wt.-% of κ-carrageenan.

Typically, the moisture content of the starting material is adjusted by adding water before feeding it into the processing aggregate. Since, in the invention, the starting material should be processed through an extruder, especially a twin-screw extruder, it is considered advantageous to feed the water into the first stage of the extrusion system.

In general, a moisture content of the starting material of 50 to 95 wt.-%, especially 60 to 85 wt.-% based on the total weight is preferred in the invention.

If it is intended to process blends of carrageenan and other components such as soy proteins or konjac with the present process, it is possible to prepare such blends without any problems before feeding them into the extruder.

The actual mixing of the starting material which, according to the invention, contains a starch and/or a hydrocolloid and/or a protein as additional components is preferably carried out at temperatures between 30 and 130° C. and especially preferred follows an increasing temperature profile in the direction of conveyance; mass pressures of ≧2 bar and especially between 4 and 40 bar are also preferred. Pressures of up to 60 or even 80 bar may also be suitable if this does not affect the product qualities to be achieved.

Typically, the claimed process is used for treating and/or processing a carrageenan-containing starting material with the aid of an extruder in such a manner that the carrageenan-containing material or an analogous starting blend mainly comprising carrageenan is mixed in the dry state in an intermittent fluid mixer for five minutes; Then, 30 to 70 wt.-% of distilled water is added at room temperature (approx. 20° C.) and mixing continued for another five minutes and finally, the mixture thus obtained is left to rest in the fluid mixer at room temperature for approx. one hour before being fed into the extrusion system.

This is usually done with the aid of a gravimetric screw dosage system, where, if necessary, additional liquid may be added to one of the first heating zones of the extruder. In general, process parameters such as the liquid content, the temperature and the shear forces influence the result of the extrusion or compounding step.

Conveyance of the carrageenan-containing material in the extruder system is usually accompanied by a temperature increase to 30 to 60° C., and then mixing and kneading should be carried out at 50 to 130° C. Circumferential speeds of the screw between 0.07 and 0.20 m·sec⁻¹ have proven to be particularly useful, with such circumferential speeds corresponding to an extruder with a screw diameter of 25 mm. Screw rotational speeds between 60 and 150 min⁻¹ are also recommended.

The total water content which is composed of the individual water contents of all the materials processed in the extruder, including the added water and the moisture content of the starting material as well as the water resulting from the humidification stage should be between 50 and 95% of the total weight.

Carrying out the proposed process is not limited to any special extruder type, but as already mentioned, a twin-screw extruder is preferred, because it guarantees better heat transfer and an improved mixing and structuring of the material may be achieved with this type.

In cooperation with the extruder cylinder, the screw thread builds up the mass pressure which is necessary to overcome the extrusion tool resistance and to pass non-conveying screw elements (shear components, kneading blocks, distributive mixing components). After that, the extruded strands are cooled in the hydrated state, cut into small pieces with the aid of a cutting mill, dried in a vacuum screen dryer and then ground.

On the whole, care must be taken that drying and grinding of the final product is carried out under such conditions that the properties of the final product are not affected in the least.

Owing to its surprisingly varied and beneficial product properties which, in particular, are achieved by the process also claimed by the present invention and which especially relate to the gelatinisation properties and a markedly decreased syneresis, the product preferably obtained by extrusion may be used in numerous areas of application where improved gelatinisation is desired, which is especially the case in the meat processing industry.

This is one of the main reasons why the present invention also includes the use of the carrageenan-containing composition with improved gelatinisation properties, said composition being used as a formulation in the food industry and/or the pharmaceutical industry, especially in gels containing water. Preferred pertinent applications are as a texturing agent, as a viscosifier, as a gelatinisation agent, as a film-forming agent, as a Theological aid and as a stabiliser.

On the whole, the present invention provides a new carrageenan-containing composition with improved gel strength, a markedly reduced syneresis and accelerated gelatinisation capabilities.

In addition, the claimed composition in powder form displays improved dispersibility in solutions, with all of these beneficial properties of the products being achieved not least by the process for improving the gelatinisation characteristics which is also claimed.

The following examples illustrate these highly surprising product features of the claimed composition which, for example, may be obtained by the process also claimed.

### EXAMPLES

A Berstorff twin-screw extruder, type ZE25-HD=48, was used for the following examples, which was coupled with a gravimetric dosing system (K-Tron or AG type T20). The amounts of liquid (distilled water) required in each case were fed into the third cylinder compartment of the extruder with the aid of a membrane pump (Prominent AKTRIEB G/4-W).

All of the starting material was either premixed in the dry state or mixed in a fluid mixer, type Günther Papenmeier TGHK-8. Humidifidation of the individual blends with distilled water was carried out with an injection needle in the fluid mixer.

If necessary, the product strands leaving the extruder were cut into pellets of approx. 4 mm in length with a strand granulator (by the Scheer company).

As a rule, the material leaving the extruder was dried in hot air in a vacuum screen dryer (Heraeus electronic) at 55° C. for 24 hours.

The characteristics of the products thus obtained were determined on the basis of their ability to form gels in salt water. For this purpose, 2.5 wt.-% of sodium chloride and sodium tripolyphosphate and then 5 wt.-% of the product concerned were placed into distilled water.

With the aid of a manometer (for example, a TAXT2 Rheometer) the gel strengths and the deformation characteristics of the gels (breaking point, flexibility and resisting force) were measured.

The breaking strength was determined in a compaction test where a load is applied to a gel disk of 12.7 mm diameter in a crystallisation dish with a penetration rate of 0.5 mm/sec. As a starting material for the gel, 1.5 wt.-% of a carrageenan powder were kept in a saline solution at 10° C. for 16 hours. After that the measurement was taken. The Theological parameters were the strength in 2 and 4 mm depth of the gel disk and the breaking point. In this compaction test, the stamp penetrates into the sample depending on the gel strength formed, overcoming the surface force, with, depending on the gel strength or breaking strength, respectively, a certain force having to be applied which is determined as “g” in 2 or 4 mm of gel depth (1 N=102 g) and which shows the breaking strength or gel strength, respectively.

Syneresis was determined in each case by keeping the gels in a crystallisation dish in a refrigerator for three days. The water released was then absorbed by blotting paper to dry the gels, and syneresis was calculated with the following formula:

$$S = \frac{{w}_{1} - {w}_{2}}{{w}_{1}} \times 100$$

wherein
w₁=weight of the moist gel,
w₂=weight of the gel carefully dried with blotting paper.

As a result, the viscosities were recorded against the temperature profile of the relevant examples by showing the so-called “Brabender graph”.

In addition, changes in the particles of the blends were determined microscopically with special staining methods, with significant differences between extruded and non-extruded blends being found:

When carrageenan obtained conventionally was stained, elongated purple particles appeared, whereas soy proteins are light-blue and have a more spherical shape.

In Lugols solution, the carrageenan components are transparent, while the konjac particles are stained purple/red.

After the extrusion process, it was not possible to clearly distinguish between the different components.

### Example 1

Starting material: κ-carrageenan from Eucheuma cottonii.

Before the extrusion process, 40 wt.-% of distilled water were added.

Extrusion conditions:
Liquid added to cylinder compartment 3; rate 0.75 kg/h
Rotational speed of the screw: n=100 min⁻¹
Dosage rate: 2 kg/h

Temperature profile:

| T1 | T2 | T3 | T4 | T5 | T6 | T7 | T8 | T9 | T10 |
|---|---|---|---|---|---|---|---|---|---|
| 35 | 40 | 70 | 90 | 90 | 90 | 90 | 90 | 80 | 80 |

Pressure at the tool inlet: 16 bar
Total moisture content of the final product: 60 wt.-%
Throughput: 2.25 kg/h

Result

| | | Breaking |
|---|---|---|
| | Syneresis (wt.-%) | strength (g) |
| Starting material | 3.8 | 476 |
| Extruded product | 2.9 | 650 |

The result showed a significant decrease of the syneresis of the gel formed with an extruded carrageenan according to the invention.

### Example 2

Starting material: κ-carrageenan from Eucheuma cottonii.

Before the extrusion process, 60 wt.-% of distilled water were added.

Extrusion conditions:
Additional liquid added to cylinder compartment 3; rate 2.5 kg/h
Rotational speed of the screw: n=100 min⁻¹
Dosage rate: 1.5 kg/h

Temperature profile:

| T1 | T2 | T3 | T4 | T5 | T6 | T7 | T8 | T9 | T10 |
|---|---|---|---|---|---|---|---|---|---|
| 35 | 40 | 90 | 130 | 130 | 130 | 130 | 130 | 120 | 120 |

Pressure at the tool inlet: 3 bar
Total moisture content of the final product: 85 wt.-%
Throughput: 4 kg/h

Result

| | Syneresis | Expandability | Breaking |
|---|---|---|---|
| | (wt.- %) | (mm) | strength (g) |
| Starting material | 3.8 | 4.8 | 476 |
| Extruded product | 2.7 | 4.8 | 710 |

The result showed a significant increase of the breaking strength of the gel formed with an extruded carrageenan according to the invention.

### Example 3

Starting material: Blend of a κ-carrageenan from Eucheuma cottonii and a soy protein isolate (F940 by DuPont).

Before the extrusion process, 80 wt.-% of carrageenan was mixed with 20 wt.-% of the soy protein in the dry state and then 60 wt.-% of distilled water were added.

Extrusion conditions:
Rotational speed of the screw: n=100 min⁻¹
Dosage rate: 2 kg/h

Temperature profile:

| T1 | T2 | T3 | T4 | T5 | T6 | T7 | T8 | T9 | T10 |
|---|---|---|---|---|---|---|---|---|---|
| 35 | 35 | 55 | 70 | 100 | 110 | 110 | 100 | 90 | 90 |

Pressure at the tool inlet: 18 bar
Total moisture content of the final product: 60 wt.-%
Throughput: 2 kg/h

Result

| | Expandability | Breaking | Syneresis |
|---|---|---|---|
| | (mm) | strength (g) | (wt.- %) |
| Starting material | 2.9 | 270 | 2 |
| Extruded product | 2.7 | 440 | 1.2-1.7 |

These results clearly show that the extrusion process mitigated the otherwise usual negative impact of the soy protein on the gel strength and at the same time, a decrease of syneresis and a significant increase of the gel strength (breaking strength) were observed in the gels of the invention containing carrageenan and protein.

### Example 4

Starting material: Blend of a κ-carrageenan from Eucheuma cottonii and konjac flour (hydrocolloid).

Before the extrusion process, 97 wt.-% of carrageenan was mixed with 3 wt.-% of konjac flour in the dry state and then 20 wt.-% of distilled water was added.

Extrusion conditions:
Additional liquid was added to the cylinder compartment 3; rate 3 kg/h
Rotational speed of the screw: n=100 min⁻¹
Dosage rate: 1 kg/h

Temperature profile:

| T1 | T2 | T3 | T4 | T5 | T6 | T7 | T8 | T9 | T10 |
|---|---|---|---|---|---|---|---|---|---|
| 35 | 35 | 50 | 70 | 80 | 80 | 70 | 60 | 60 | 50 |

Pressure at the tool inlet: 4 bar
Total moisture content of the final product: 80 wt.-%
Throughput: 4 kg/h

Result

| | Expandability | Breaking | Syneresis |
|---|---|---|---|
| | (mm) | strength (g) | (wt.- %) |
| Starting material | 3.4 | 515 | 1.4 |
| Extruded product | 3.8 | 885 | 1.0 |

These results show that the positive influence of konjac on the properties of the carrageenan gel may be further improved by the extrusion process and that a decrease of the syneresis and a significant increase of the gel strength may be observed in the gel of the invention containing carrageenan and konjac.

### Example 5

Starting material: Blend of a κ-carrageenan from Eucheuma cottonii and a soy protein isolate (F940 by DuPont).

Before the extrusion process, 80 wt.-% of carrageenan was mixed with 20 wt.-% of the soy protein in the dry state and then 60 wt.-% of distilled water were added.

Extrusion conditions:
Additional liquid was added to the cylinder compartment 3; rate 3.33 kg/h
Rotational speed of the screw: n=100 min⁻¹
Dosage rate: 2 kg/h

Temperature profile:

| T1 | T2 | T3 | T4 | T5 | T6 | T7 | T8 | T9 | T10 |
|---|---|---|---|---|---|---|---|---|---|
| 35 | 35 | 55 | 70 | 100 | 110 | 110 | 100 | 90 | 90 |

Pressure at the tool inlet: 2 bar
Total moisture content of the final product: 85 wt.-%
Throughput: 5.33 kg/h

Result

| | Syneresis | Breaking | Gelatinisation |
|---|---|---|---|
| | (wt.- %) | strength (g) | temperature (° C.) |
| Starting | 2.1 | 290 | 30 |
| material | | | |
| Extruded | 1.3 | 490 | 38 |
| product | | | |

In the extruded product according to the invention, the gelatinisation step takes place at a temperature which is increased by 8° C. over that of the starting material.

### Example 6

Starting material: Blend of a κ-carrageenan from Eucheuma cottonii and 1) a soy protein isolate (20 wt.-%) or 2) konjac flour (30 wt.-%).

Result:

| | Microscopic observation |
|---|---|
| Starting material | Two separate components |
| Extruded products | The soy particles or the konjac particles, |
| 1) and 2) respectively | respectively, are encapsulated in the carrageenan |
| | particles. |

On the whole, it was found that the konjac particles were encapsulated in the carrageenan particles in the first case, said konjac particles being much smaller than those of the starting material. In the second case, it was no longer possible to detect the soy protein particles under the microscope; they seem to have been absorbed completely by the carrageenan particles.

1. A carrageenan-containing composition, characterised in that it has a syneresis of ≦3.0 wt.-% and an improved breaking strength of at least 20% when compared with the carrageenan-containing starting material and consists of at least 60 wt.-% of κ-carrageenan.

2. A composition according to claim 1, characterised in that the starting material has a carrageenan content between 2 and 10 wt.-%, especially from 4 to 7 wt.-%.

3. A composition according to claim 1, characterised in that the syneresis is ≦2.0 wt.-%.

4. A composition according to claim 1, characterised in that it has an improved breaking strength of at least 40% when compared with the carrageenan-containing starting material.

5. A composition according to claim 1, characterised in that it additionally contains a starch and/or a hydrocolloid and/or a protein.

6. A composition according to claim 5, characterised in that the starch is one selected from corn, maize, potato, wheat, tapioca, rice or mixtures thereof.

7. A composition according to claim 6, characterised in that the hydrocolloid is derived from the series guar, locust beans, karaya, konjac, cellulose, cellulose ether, microcrystalline cellulose, xanthan gum, pectin, alginates, agar or mixtures thereof.

8. A composition according to claim 5, characterised in that the protein is one of vegetable origin, especially from leguminous plants, particularly soy.

9. A composition according to claim 1, characterised in that it is a product subjected to treatment under high shear forces and high pressure.

10. A process for the treatment of a carrageenan-containing starting material consisting of at least 60 wt.-% of κ-carrageenan, characterised in that the starting material is first humidified, then subjected to intensive shearing and mixing at elevated temperatures and under conditions of increased pressure and, finally, the mass obtained is cooled and ground.

11. A process according to claim 10, characterised in that the starting material is humidified with water.

12. A process according to claim 10, characterised in that the starting material is humidified to a moisture content between 50 and 95 wt.-%, especially 60 to 85 wt.-%, based on the total weight.

13. A process according to claim 10, characterised in that shearing and mixing is carried out at temperatures between 30 and 130° C.

14. A process according to claim 13, characterised in that shearing and mixing is carried out under a temperature profile increasing in the direction of conveyance.

15. A process according to claim 10, characterised in that the pressure of the mass is ≧2 bar and preferably between 4 and 40 bar.

16. A process according to claim 10, characterised in that shearing and mixing is carried out with the aid of an extruder and preferably with the aid of a twin-screw extruder.

17. A carrageenan-containing composition having a syneresis of ≦3.0 wt.-% and an improved breaking strength of at least 20% when compared with the starting material having a carrageenan content between 2 and 10 wt.-% which is obtainable by a process according to claim 10.

18. The use of a carrageenan-containing composition according to claim 1 or a carrageenan-containing composition having a syneresis of ≦3.0 wt.-% and an improved breaking strength of at least 20% when compared with the starting material having a carrageenan content between 2 and 10 wt.-% as a formulation in the food industry and/or for pharmaceutical purposes.

19. The use according to claim 18, characterised in that the carrageenan-containing composition is used in gels containing water.

20. The use according to claim 18, characterised in that the composition is used as a texturing agent, an agent influencing viscosity, a gelling agent, a film-forming agent, a Theological aid or as a stabiliser.
